(19) United States
(12) Patent Application Publication
(10) Pub. No.: US 2007/0299123 A1
(43) Pub. Date: Dec. 27, 2007

(54) Amorphous frovatriptan succinate and process for the preparation thereof

(75) Inventors: Samir Jaivant Naik, Thane (IN); Anthony Melvin Crasto, Thane (IN); Narendra Sriram Joshi, Navi Mumbai (IN); Sachin Srivastava, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/823,446

(22) Filed: Jun. 27, 2007

(30) Foreign Application Priority Data
Jun. 27, 2006 (IN) 1010/MUM/2006

(51) Int. Cl. A61K 31/403; C07D 209/82

(52) U.S. Cl. 514/411; 548/444

(57) ABSTRACT

Frovatriptan succinate substantially in an amorphous form is disclosed. Also disclosed is a composition comprising frovatriptan succinate in a solid form, wherein at least about 80% by weight of the solid is frovatriptan succinate in an amorphous form. The present invention also provides a process for preparing frovatriptan succinate substantially in an amorphous form by providing a solution of frovatriptan succinate in one or more solvents capable of dissolving the frovatriptan succinate and substantially removing the solvent from the solution.

### PRIORITY

This application claims the benefit under 35 U.S.C. §119 to Indian Provisional Application 1010/MUM/2006, filed on Jun. 27, 2006, the contents of which are incorporated by reference herein.

### BACKGROUND OF THE INVENTION

1. Technical Field

The present invention generally relates to frovatriptan succinate substantially in an amorphous form and a process for its preparation. More particularly, the present invention generally relates to a composition comprising frovatriptan succinate in a solid form, wherein at least 80% by weight of the solid frovatriptan succinate is in amorphous form.

2. Description of the Related Art

Frovatriptan succinate, also known as (R)-(+)-6-carboxamido-3-N-methylamino-1,2,3,4-tetrahydrocarbazole, has the following structure: 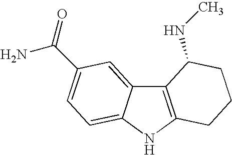
Frovatriptan succinate is a 5HT₁ receptor agonist. It is marketed under the name FROVA® for the treatment of migraine.

WO 93/00086 discloses a group of tetrahydrocarbazole derivatives, which have activity as 5HT₁ receptor agonists, and are therefore useful in the treatment of migraines. A specific compound disclosed therein is 3-methylamino-6-carboxamido-1,2,3,4-tetrahydrocarbazole hydrochloride prepared by a six stage process, via 3-methylamino-6-cyano-1,2,3,4-tetrahydrocarbazole.

WO 94/14772 discloses enantiomers of certain carbazole derivatives, including the aforementioned compound (R)-(+)-6-carboxamido-3-N-methylamino-1,2,3,4-tetrahydrocarbazole, salts and solvates thereof. WO 94/14772 further discloses various methods by which single enantiomers can be prepared; one of which involves:
(a) separation of an enantiomeric mixture of the compound or a derivative thereof by chromatography, e.g. on a chiral HPLC column;
(b) alkylation of (+) or (−) enantiomer of 3-amino-6-carboxamido-1,2,3,4-tetrahydrocarbazole or a salt thereof; and
(c) separation of diastereoisomers of a chiral derivative e.g. a chiral salt of the compound by crystallization or chromatography.

Amorphous materials do not exhibit the three-dimensional long-range order found in crystalline materials, but is structurally more similar to liquids where the arrangement of molecules is random. Amorphous solids do not give a definitive x-ray diffraction pattern (XRD). In addition, amorphous solids do not give rise to a melting point and tend to liquefy at some point beyond the glass transition point. Because amorphous solids do not have lattice energy, they usually dissolve in a solvent more rapidly and consequently may provide rapid bioavailability. Furthermore, amorphous forms of a drug may offer significant advantages over crystalline forms of the same drug in solid dosage form manufacture process such as compressibility, economically or environmentally suitable solvents or process, or higher purity or yield of the desired product.

Consequently, it would be a significant contribution to the art to provide an amorphous form of frovatriptan succinate of superior quality for use in a pharmaceutical preparation.

### SUMMARY OF THE INVENTION

In accordance with one embodiment of the present invention, frovatriptan succinate in an amorphous form is provided.

In accordance with a second embodiment of the present invention, a composition is provided comprising frovatriptan succinate in a solid form, wherein at least about 80% by weight of the solid frovatriptan succinate in an amorphous form.

In accordance with a third embodiment of the present invention, a process for preparing frovatriptan succinate in an amorphous form is provided, the process comprising the steps of:
(a) providing a solution of frovatriptan succinate in one or more solvents capable of dissolving the frovatriptan succinate; and
(b) substantially removing the solvent from the solution to provide amorphous frovatriptan succinate.
   Definitions

The term “compound” shall be understood to mean a chemical substance that includes molecules of the same chemical structure.

The term “pharmaceutically acceptable” shall be understood to mean that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds.

The term “pharmaceutical composition” shall be understood to encompass a product comprising the active ingredient(s) and pharmaceutically acceptable excipients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, any additional active ingredient(s), and pharmaceutically acceptable excipients.

The term “isolating” shall be understood to indicate separation of the compound being isolated regardless of the purity of the isolated compound from any unwanted substance, which presents with the compound as a mixture. Thus, degree of the purity of the isolated or separated compound does not affect the status of “isolating.”

### DETAILED DESCRIPTION OF THE PREFERRED EMBODIMENT

The present invention provides frovatriptan succinate substantially in an amorphous form. The amorphous solid of frovatriptan succinate can be prepared by a process involving (a) providing a solution of frovatriptan succinate in one or more solvents capable of dissolving the frovatriptan succinate succinate; (b) optionally, filtering the solvent solution to remove any extraneous matter; and (c) substantially removing the solvent from the solution to provide frovatriptan succinate substantially in an amorphous form.

Step (a) of the process of the present invention includes dissolving any form of frovatriptan succinate in a suitable solvent or obtaining an existing solution from a previous processing step. Suitable solvents include, but are not limited to, alcoholic solvents having from 1 to 6 carbon atoms, aromatic solvents, non-aromatic solvents and the like and mixtures thereof. The dissolution can be carried out at a temperature ranging from about 20° C. to about 100° C. or at the reflux temperature of the solvent used.

The clear solution can be optionally filtered to remove any extraneous matter present in the solution using any standard filtration techniques known in the art.

Step (c) of the process of the present invention can be carried out by, for example, substantially complete evaporation of the solvent, concentrating the solution, cooling the solution to obtain amorphous form and filtering the solid under an inert atmosphere. Evaporation can be achieved at sub-zero temperatures by a lyophilisation or freeze-drying technique. The solution may also be completely evaporated in, for example, a pilot plant rota vapor, a vacuum paddle dryer or in a conventional reactor under vacuum above about 720 mm Hg by flash evaporation techniques by using an agitated thin film dryer (“ATFD”), or evaporated by spray drying to obtain a dry amorphous powder.

A preferred method to remove the solvent involves spray-drying, in which a solution of frovatriptan succinate is sprayed into the spray drier at the flow rate ranging from about 10 to about 300 ml/hr, and preferably a flow rate of about 40 to about 200 ml/hr. The air inlet temperature to the spray drier used may range from about 25° C. to about 150° C., preferably from about 60° C. to about 110° C. and the outlet air temperature used may range from about 30° C. to about 90° C. Of course, specific conditions will vary somewhat for spray drying using different equipment configurations. The solid residue obtained after the solvent removal is isolated and, if desired, can be dried further using conventional methods. The advantages of the process include simplicity, eco-friendliness and suitability for commercial use.

Another preferred method for removing the solvent is using vertical agitated thin-film drying (or evaporation). Agitated thin film evaporation technology involves separating the volatile component using indirect heat transfer coupled with mechanical agitation of the flowing film under controlled condition. In a vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The frovatriptan succinate substantially in an amorphous form thus obtained may be further dried in, for example, a vacuum tray dryer, rotocon vacuum dryer, vacuum paddle dryer or pilot plant rota vapor, to further lower residual solvents.

Another aspect of the present invention provides amorphous frovatriptan succinate having a chemical purity of at least about 96% as measure by HPLC, preferably at least about 99%, and more preferably at least about 99.5%. Moreover, the highly purified amorphous frovatriptan succinate may be obtained substantially free of any unknown impurity, e.g., a content of less than about 0.1% of impurities.

Yet another aspect of the present invention provides a composition comprising frovatriptan succinate in solid form, wherein at least about 80% by weight of the solid frovatriptan succinate is an amorphous form. The remainder of the solid frovatriptan succinate in the composition, i.e., about 20% or less of the total weight of frovatriptan succinate, may be other forms of frovatriptan succinate, e.g., a crystalline form. In another embodiment of the invention, the composition may include at least about 95% of the amorphous form of frovatriptan succinate with respect to total weight of the solid frovatriptan succinate in the composition. In another embodiment of the invention, the composition may include at least about 99% of the amorphous form of frovatriptan succinate with respect to total weight of the solid frovatriptan succinate in the composition.

In addition to X-ray powder diffraction, amorphous frovatriptan succinate, or the presence of some amorphous frovatriptan succinate, can be distinguished from crystalline frovatriptan succinate, using Raman spectroscopy, solution calorimetry, differential scanning calorimetry, solid state nuclear magnetic resonance spectra (ssNMR) or infra-red spectroscopy. Each of these techniques is well established in the art. Amorphous frovatriptan succinate can also be identified based on the morphology of the particles seen under an electron microscope.

Yet another aspect of the present invention is directed to pharmaceutical compositions containing at least the novel frovatriptan succinate substantially in an amorphous form of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The novel frovatriptan succinate substantially in an amorphous form of the present invention also may be administered as suppositories, ophthalmic ointments and suspensions, and parenteral suspensions, which are administered by other routes. The dosage forms may contain the novel frovatriptan succinate substantially in an amorphous form of the present invention as is or, alternatively, may contain the frovatriptan succinate substantially in an amorphous form thereof of the present invention as part of a composition as, for example, a composition disclosed above. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants.

Capsule dosages will contain the novel frovatriptan succinate substantially in an amorphous form of the present invention within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants such as magnesium and calcium stearate and sodium stearyl fumarate; flavorings; sweeteners; preservatives; pharmaceutically acceptable dyes and glidants such as silicon dioxide.

In one embodiment, the frovatriptan succinate substantially in an amorphous form of the present invention for use in the pharmaceutical compositions of the present invention can have a D₅₀ and D₉₀ particle size of less than about 300 microns, preferably less than about 200 microns, more preferably less than about 150 microns, still more preferably less than about 50 microns and most preferably less than about 10 microns. The particle sizes of the novel frovatriptan succinate substantially in an amorphous form of the present invention can be obtained by, for example, any milling, grinding, micronizing or other particle size reduction method known in the art to bring the solid state frovatriptan succinate substantially in an amorphous form of the present invention into any of the foregoing desired particle size range.

Actual dosage levels of the frovatriptan succinate substantially in an amorphous form of the present invention may be varied to obtain an amount of the amorphous form of frovatriptan succinate that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the frovatriptan succinate substantially in an amorphous form of the present invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. A pharmaceutical composition of the invention will generally include about 0.1% by weight to about 99% by weight of active ingredient, and preferably about 1% by weight to 50% by weight.

Further, the frovatriptan succinate substantially in an amorphous form of the present invention can be used in a method for treatment of pain related condition such as the treatment of migraines. The method of treatment includes administering to a mammal in need of treatment a dosage form that includes a therapeutically effective amount of the frovatriptan succinate substantially in an amorphous form of the present invention.

The following examples are provided to enable one skilled in the art to practice the invention and are merely illustrative of the invention. The examples should not be read as limiting the scope of the invention as defined in the features and advantages.

### EXAMPLE 1

Preparation of Amorphous Frovatriptan Succinate by Spray Drying.

Frovatriptan succinate (5 g) is dissolved in an alcoholic solvent such as methanol (700 ml) in a round bottom flask. The solution is filtered through a filtration medium or filter aid to remove any extraneous matter. The filtrate is then spray dried at 40 to 45° C. until the solvent is completely removed. The solid obtained is collected and dried under high vacuum at a suitable temperature and a free flowing amorphous solid is obtained.

### EXAMPLE 2

Preparation of Amorphous Frovatriptan Succinate by Vertical Agitated Thin Film Drying.

Frovatriptan succinate (5 g) and an alcoholic solvent such as methanol (500 ml) are charged into a clean and dry round bottom flask and stirred for about 30 minutes. The solution is filtered through a filtration medium or filter aid to remove any extraneous matter. The filtrate is subjected to agitated thin film drying with a feed rate of about 5 L/hour, under a reduced pressure of about 5-20 torr and a jacket temperature of about 35-40° C. The solid obtained is collected and dried under high vacuum at a suitable temperature and a free flowing amorphous solid is obtained.

### EXAMPLE 3

Preparation of Amorphous Frovatriptan Succinate by Lyophilisation.

Frovatriptan succinate (5 g) is dissolved in an alcoholic solvent such as methanol (700 ml) at room temperature. The solution is filtered through a filtration medium or filter aid to remove any extraneous matter. The filtrate is subjected to lyophilisation until a free flowing amorphous solid is obtained.

While the above description contains many specifics, these specifics should not be construed as limitations of the invention, but merely as exemplifications of preferred embodiments thereof. Those skilled in the art will envision many other embodiments within the scope and spirit of the invention as defined by the features and advantages appended hereto.

What is claimed is:

1. Frovatriptan succinate in an amorphous form.

2. A composition comprising frovatriptan succinate in a solid form, wherein at least about 80% by weight of the solid is frovatriptan succinate in an amorphous form.

3. The composition of claim 2, wherein the frovatriptan succinate has a chemical purity of about 96%.

4. The composition of claim 2, wherein the frovatriptan succinate has a chemical purity of about 99%.

5. The composition of claim 2, wherein the frovatriptan succinate has a chemical purity of about 99.5%.

6. The composition of claim 2, wherein the frovatriptan succinate has a D₅₀ particle size of less than about 300 microns.

7. The composition of claim 2, wherein the frovatriptan succinate has a D₅₀ particle size of less than about 200 microns.

8. The composition of claim 2, wherein the frovatriptan succinate has a D₅₀ particle size of less than about 150 microns.

9. The composition of claim 2, wherein the frovatriptan succinate has a D₅₀ particle size less than about 50 microns.

10. The composition of claim 2, wherein the frovatriptan succinate has a D₅₀ particle size less than about 10 microns.

11. The composition of claim 2, wherein the frovatriptan succinate has a D₉₀ particle size of less than about 300 microns.

12. The composition of claim 2, wherein the frovatriptan succinate has a D₉₀ particle size of less than about 200 microns.

13. The composition of claim 2, wherein the frovatriptan succinate has a D₉₀ particle size of less than about 150 microns.

14. The composition of claim 2, wherein the frovatriptan succinate has a D₉₀ particle size less than about 50 microns.

15. The composition of claim 2, wherein the frovatriptan succinate has a D₉₀ particle size less than about 10 microns.

16. A pharmaceutical composition comprising a therapeutically effective amount of the frovatriptan succinate in an amorphous form of claim 1 and at least one pharmaceutical excipient.

17. A pharmaceutical composition comprising a therapeutically effective amount of the composition of claim 2 and at least one pharmaceutical excipient.

18. A pharmaceutical composition comprising a therapeutically effective amount of the composition of claim 3 and at least one pharmaceutical excipient.

19. A process for preparing frovatriptan succinate substantially in an amorphous form, the process comprising:
(a) providing a solution of frovatriptan succinate in one or more solvents capable of dissolving the frovatriptan succinate; and
(b) substantially removing the solvent from the solution to provide amorphous frovatriptan succinate.

20. The process of claim 19, wherein the solvents are selected from the group consisting of an alcoholic solvent having from 1 to 6 carbon atoms, aromatic solvent, non-aromatic solvent and combinations thereof.
